(19) United States
(12) Patent Application Publication
(10) Pub. No.: US 2022/0054322 A1
(43) Pub. Date: Feb. 24, 2022

(54) MEDICAL COMPRESSION STOCKINGS

(71) Applicant: The Hong Kong Research Institute of Textiles and Apparel Limited, Hong Kong (CN)

(72) Inventors: Xiaoming Tao, Hong Kong (CN); Ying Xiong, Hong Kong (CN); Jinyun Zhou, Hong Kong (CN); Shirui Liu, Hong Kong (CN); Chad Cheuk-Wa Tse, Hong Kong (CN); Xiuyuan Hu, Hong Kong (CN)

(73) Assignee: The Hong Kong Research Institute of Textiles and Apparel Limited, Hong Kong (CN)

(21) Appl. No.: 17/432,860

(22) PCT Filed: Feb. 25, 2019
(86) PCT No.: PCT/CN2019/076077

§ 371 Date: Aug. 20, 2021

(51) Int. Cl. A61F 13/08

(52) U.S. Cl. CPC A61F 13/08

(57) ABSTRACT

Disclosed is a pair of medical compression stocking, each comprising a body, an auxiliary pressure part and a present sensing part. The body is formed by means of knitting knitted yarns and inlay yarns. The auxiliary pressure part is integrated with the body, is located at an ankle of a lower limb of each medical compression stocking, and can be in contact with an ankle of a wearer. The pressure sensing part comprises a conducting part and a signal receiving part, wherein the conducting part is integrated with the body and is used for sensing pressure at a corresponding part of the medical compression stocking; and the signal receiving part can receive a signal from the conducting part, and same determines the value of the pressure from the corresponding part according to the signal.

### CROSS-REFERENCE TO RELATED APPLICATION

This application is the 371 application of PCT Application No. PCT/CN2019/076077, filed Feb. 25, 2019, the entire contents of which are incorporated herein by reference.

### TECHNICAL FIELD

The present disclosure relates to the technical field of compression stockings, and in particular to medical compression stockings.

### BACKGROUND

Medical compression stockings (MCSs) are an important component for treatment of varicose veins. Medical compression stockings are designed to gradually reduce pressure from a lower part to an upper part of a limb, thereby increasing flow rate of venous, reducing swelling of vein wall, improving valve function, and helping to improve return of venous blood from a distal end to a proximal end.

Compression stockings are generally knitted structures, which are composed of knitting yarn and inlay yarn. By adjusting parameters of knitting process, inlay yarn and structure, compression stockings with different pressure levels, sizes and shapes can be manufactured for different users.

However, since a cross-sectional shape of the lower limb is irregular, that is, from the lower part to the upper part, the cross-sectional shape of the lower limb is changed from an irregular shape to an ellipse. For the irregular shape, compression stockings cannot apply uniform and accurate pressure to the lower part of the limb. Especially at an ankle, the pressure of the compression stockings cannot be applied to position close to bones. There is hollow area between the compression stockings and the ankle. Therefore, the pressure on the ankle side is insufficient, which is much less than the pressure on other parts, causing patients with venous insufficiency to be often prone to ulcerate here.

### SUMMARY

Based on the above problems, the present disclosure provides medical compression stockings, which can optimize pressure distribution of legs, especially at ankle, and can monitor the pressure.

To achieve the above object, the present disclosure provides medical compression stockings, which includes a main body, an auxiliary pressure part and a pressure sensing part. The main body is formed by knitting yarn and inlay yarn. The auxiliary pressure part is integrated with the main body and located at an ankle of a lower limb of the medical compression stockings, wherein the auxiliary pressure part is configured to be in contact with an ankle of a wearer. The pressure sensing part includes a conductive part and a signal receiving part, wherein the conductive part is integrated with the main body to sense pressure of corresponding part of the medical compression stockings, and the signal receiving part is configured to receive a signal from the conductive part and determine a pressure value of the corresponding part according to the signal.

According to an embodiment, the knitting yarn and/or the inlay yarn are single-layer elastic wrap yarn.

According to an embodiment, the knitting yarn and/or the inlay yarn are double-layer elastic wrap yarn.

According to an embodiment, the number of auxiliary pressure parts is one or more, and the auxiliary pressure part is configured to directly contact a medial malleolus of the wearer.

According to an embodiment, the auxiliary pressure part includes a terry structure, and the terry structure is integrated with the main body by embroidering or knitting process.

According to an embodiment, knitting yarn corresponding to the auxiliary pressure part is formed into a plurality of continuous tuck structures using a tuck method.

According to an embodiment, along a warp direction, there are 2 to 3 tuck stitches continuously arranged, and along a weft direction, there are 3 to 5 tuck stitches continuously arranged.

According to an embodiment, material of the terry structure is natural fiber or man-made fiber.

According to an embodiment, the auxiliary pressure part includes a gasket, and the gasket is sewn on the medial malleolus of a lower limb of the medical compression stockings.

According to an embodiment, material of the gasket is silicone, the auxiliary pressure part further includes auxiliary fabrics, the silicone is sandwiched between the main body and the auxiliary fabrics, area of the auxiliary fabric is greater than or equal to area of the silicone, and the auxiliary fabric is in direct contact with the medial malleolus of the wearer.

According to an embodiment, the conductive part includes a flexible conductive part, the flexible conductive part is sewn into the main body to be integrated with the main body, and the flexible conductive part senses pressure changes through changes in resistance or capacitance.

According to an embodiment, the flexible conductive part is a flexible conductive wire or a conductive elastic wrap yarn, and the flexible conductive part is sewn into the main body by means of loop formation, yarn addition or laid-in.

According to an embodiment, the number of conductive parts is one or more, and the conductive part is arranged at one or more positions of the main body.

According to an embodiment, the number of the conductive parts is multiple, and the conductive parts are arranged according to positions where pressure measurement is required.

According to an embodiment, the conductive parts are located on inner side, outer side, front side and/or back side of the lower limb of the medical compression stockings.

According to an embodiment, the conductive part further includes a connecting wire, and the flexible conductive part is electrically connected to the signal receiving part through the connecting wire.

According to an embodiment, the signal receiving part is also configured to compare a detected pressure value with a preset pressure standard threshold, and when the detected pressure value exceeds the preset pressure standard threshold value, a reminder is issued to the wearer.

Compared with related art, the present disclosure has beneficial effects: in the present disclosure, an auxiliary pressure part is integrated at an ankle of a lower limb of medical compression stockings. When worn, the auxiliary pressure part can properly fill hollow area between the medial malleolus and the tibia, and compensate the pressure of this part, so that the pressure from the compression stockings is transmitted to the ankle, so the pressure of the ankle is even, which can optimize the leg, especially the ankle, and avoid ulceration of patients with venous insufficiency. Moreover, the auxiliary pressure part is integrated with the main body, the two are inseparable, therefore, the auxiliary pressure part will not be displaced relative to the main body during wearing and putting on and off, and can always be kept at the position corresponding to the ankle of the lower limb, thus ensuring accuracy and convenience of use.

In addition, the medical compression stockings are also integrated with a pressure sensing part, so that the pressure applied by the medical compression stockings to the legs can be measured in real time, and the measured values have extremely high accuracy and reliability. Therefore, it is helpful for the wearer to monitor the comfort of the medical compression stockings.

### BRIEF DESCRIPTION OF THE DRAWINGS

FIG. 1 is a schematic diagram of wearing state of medical compression stockings according to an embodiment of the present disclosure.

FIG. 2 is a schematic diagram of wearing state of medical compression stockings according to another embodiment of the present disclosure.

FIG. 3 is a side view of wearing state of medical compression stockings according to another embodiment of the present disclosure.

FIG. 4 is a rear view of wearing state of medical compression stockings according to another embodiment of the present disclosure.

FIG. 5 is a schematic diagram of wearing state of medical compression stockings according to an embodiment of the present disclosure, which shows a plurality of conductive parts.

FIG. 6 is a partial enlargement view of part structure in FIG. 5.

FIG. 7 is a side view of wearing state of medical compression stockings according to another embodiment of the present disclosure.

FIG. 8 is a partial enlargement view of a tuck structure of part A in FIG. 7, in which inlay yarn is not shown.

FIG. 9 is a schematic diagram of textile structure of terry structure, in which inlay yarn and tuck structure are not shown.

FIG. 10 is a schematic diagram of a relationship between tensile stress and elongation of medical compression stockings. and

FIG. 11 is a schematic diagram of pressure distribution at multiple positions of medical compression stockings.

### DETAILED DESCRIPTION

Exemplary embodiments of the present disclosure will now be described more fully with reference to accompanying drawings. However, the exemplary embodiments herein are provided to assist in the understanding of the disclosure and are not intended to limit the disclosure in any way. On the contrary, the embodiments are provided to make the description of the present disclosure more comprehensive and complete, and to transmit the concept of the exemplary embodiments to those skilled in the art. In the figure, thickness of regions and layers may be exaggerated for clarity. The same reference numerals in the drawings denote the same or similar parts, and the repeated description thereof will be omitted.

Further, described features, structures, or advantages can be combined in any suitable manner in one or more embodiments. In the following description, numerous specific details are provided to give a full understanding of the embodiments of the present disclosure. However, one skilled in the art may appreciate that one or more of the specific details can be omitted while practicing technical solutions of the present disclosure, or using other equivalent methods, methods, apparatuses and steps to substitute. In other cases, well-known structures, methods, implementations, or operations are not shown or described in detail to avoid overwhelming attention and obscure various aspects of the present disclosure.

The existing compression stockings cannot apply uniform and accurate pressure to a lower part of a limb. Especially at an ankle, the pressure of the compression stockings cannot be applied to position close to bones, and there is hollow area between the compression stockings and the ankle. Therefore, the pressure on the ankle side is insufficient, which is much less than the pressure on other parts, causing patients with venous insufficiency to often ulcerate here.

An embodiment proposes following research: adding a detachable pad at the ankle of the compression stockings to achieve pressure there. By embedding the pad, the pressure at the ankle can be improved, and the pressure here is increased due to the thickness of the pad. However, since the detachable cushion part is easy to shift during wearing, it causes great inconvenience and inaccuracy during actual use.

In addition, an embodiment provides a pressure measurement system, which may include in vivo measurement and in vitro measurement. In vitro pressure measurement usually uses a test equipment to measure the pressure between compression stockings and artificial legs with metal or air bag probes. However, there is a deviation between this test method and actual contact state. Another embodiment is to perform a tension test on the compression stockings, and a formula is needed to calculate the pressure later.

In vivo pressure measurement means that the tester directly measures the pressure between the compression stockings and the leg. However, this method is inconvenient and difficult for non-professionals to implement. Therefore, so far, there has not yet been a pressure monitoring system integrated in compression stockings.

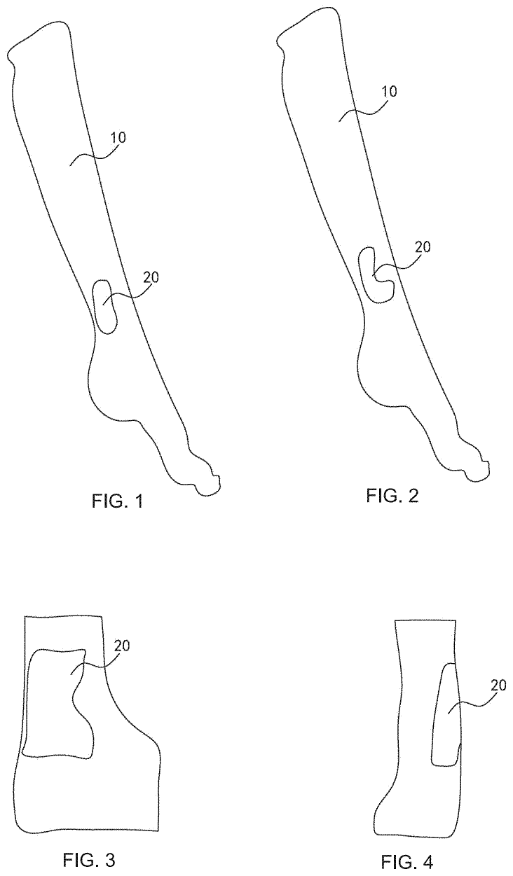

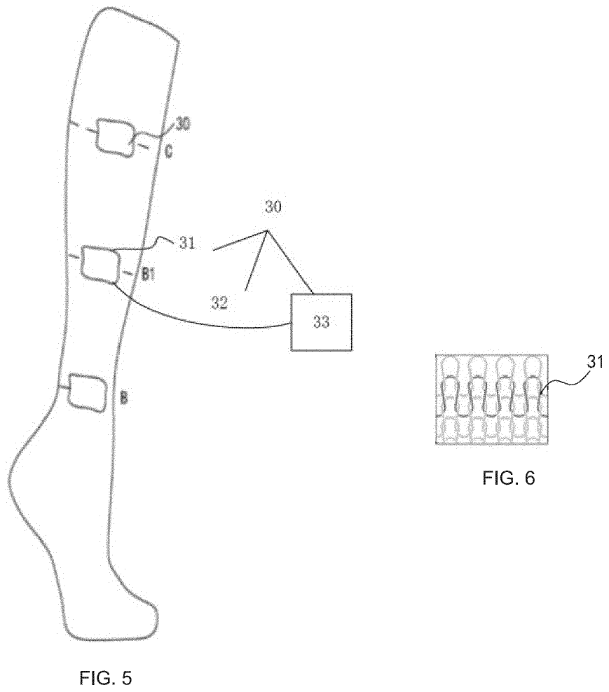

To solve the above problems, the present disclosure provides medical compression stockings, as shown in FIG. 1, FIG. 2 and FIG. 5, which includes a main body 10, an auxiliary pressure part 20 and a pressure sensing part 30. The main body 10 is formed by knitting yarn and inlay yarn. The auxiliary pressure part 20 is integrated with the main body 10 and located at an ankle of a lower limb of the medical compression stockings, wherein the auxiliary pressure part is configured to be in contact with an ankle of a wearer. The pressure sensing part 30 includes a conductive part and a signal receiving part, wherein the conductive part is integrated with the main body 10 to sense pressure of corresponding part of the medical compression stockings, and the signal receiving part is configured to receive a signal from the conductive part and determine a pressure value of the corresponding part according to the signal.

That is, in the present disclosure, an auxiliary pressure part is integrated at an ankle of a lower limb of medical compression stockings. When worn, the auxiliary pressure part can properly fill hollow area between the medial malleolus and the tibia, and compensate the pressure of this part, so that the pressure from the compression stockings is transmitted to the ankle, so the pressure of the ankle is even, which can optimize the leg, especially the ankle, and avoid ulceration of patients with venous insufficiency. Moreover, the auxiliary pressure part is integrated with the main body, the two are inseparable, therefore, the auxiliary pressure part will not be displaced relative to the main body during wearing and putting on and off, and can always be kept at the position corresponding to the ankle of the lower limb, thus ensuring accuracy and convenience of use.

In addition, the medical compression stockings are also integrated with a pressure sensing part, so that the pressure applied by the medical compression stockings to the legs can be measured in real time, and the measured values have extremely high accuracy and reliability. Therefore, it is helpful for the wearer to monitor the comfort of the medical compression stockings.

Compared with the aforementioned in vivo measurement and in vitro measurement, in the embodiment, the pressure sensing part is integrated into the medical compression stockings, which can monitor the pressure in real situations in real time, and therefore has extremely high accuracy and operability.

In an embodiment, the signal receiving part can also compare a detected pressure value with a preset pressure standard threshold, and when the detected pressure value exceeds the preset pressure standard threshold value, a reminder is issued to the wearer. The preset pressure standard threshold can be set according to the pressure standard level. Therefore, the wearer can judge whether the pressure provided by the medical compression stockings is appropriate based on the comparison between the measured pressure value and the preset pressure standard threshold. If the pressure is too high or too low, the wearer can be reminded.

In addition, in other embodiments, the pressure sensing unit 30 can also be applied to fields other than medical compression stockings, for example, the pressure sensing unit can be integrated into other compression clothing that needs to monitor pressure, and can provide assistance to patients, medical personnel, and medical manufacturers in pressure therapy.

In summary, the embodiment provides medical compression stockings integrated with the auxiliary pressure part 20 and the pressure sensing part 30, which can effectively provide pressure to the ankle and monitor pressure accurately in real time, and is convenient to use, simple in structure, and convenient manufacture.

Hereinafter, the structure of the auxiliary pressure part 20 of the medical compression stockings will be described in detail through a specific embodiment.

The main body 10 of the medical compression stockings is composed of knitting yarn and inlay yarn. In some embodiments, the heel part does not include the inlay yarn.

The knitting yarn is woven into a ground weave, and the inlay yarn is sandwiched in the ground weave in the form of a pad. Both or one of knitting yarn and inlay yarn may be a single-layer or double-layer elastic wrap yarn, and the elastic wrap yarn includes an outer layer of wrap yarn and an inner elastic yarn core.

The material of the wrap yarn is, for example, nylon, cotton, or filaments formed from bio-type PLA/PHBV materials, false twisted textured yarns or air-jet textured yarns. The material of the elastic yarn core is, for example, spandex, rubber, or other elastic filaments or yarns.

Preferably, the inner diameter of the elastic yarn core of the inlay yarn is larger than that of the knitting yarn, so as to provide greater elasticity for the medical compression stockings.

As shown in FIG. 1 to FIG. 4, the number of auxiliary pressure parts 20 is one, and it can directly contact the medial malleolus of the wearer. In some embodiments, the number of auxiliary pressure parts 20 is multiple, which can be arranged at intervals or continuously in any direction. The shape, number, and position of the auxiliary pressure part 20 can be appropriately changed as needed.

The auxiliary pressure part 20 includes a terry structure, as shown in FIG. 8 and FIG. 9, the terry structure is integrated with the main body 10 by a embroidery process.

The knitting yarn corresponding to the auxiliary pressure part 20 is formed into a plurality of continuous tuck structures using a tuck method.

That is, in the embodiment, the knitting yarn in most area of the entire medical compression stockings uses a plain stitch, and only at the position of the auxiliary pressure part 20, the knitting yarn uses a continuous tuck structure.

FIG. 9 schematically shows the structure of the auxiliary pressure part 20, wherein, white represents the knitting yarn 11 as the ground weave, black and gray yarns represent the terry structure 12, in order to clearly show the terry structure, the inlay yarn sandwiched between the knitting yarn is not shown in FIG. 9, but in fact, in the embodiment, the auxiliary pressure part 20 includes the knitting yarn, a inlay yarn and the terry structure.

In the embodiment, along the warp direction, there are 2 to 3 tuck structures continuously arranged, and along a weft direction, there are 3 to 5 tuck structures continuously arranged. Another function of the tuck structures is to make the tissue of this part of the fabric tighter and thicker, so as to improve the fit with the skin. Therefore, in other embodiments, the auxiliary pressure part 20 may be the tuck structure and does not need to include the terry structure.

It should be understood that the number and arrangement of tuck structures are only examples, and can be adjusted as needed.

The form of the terry structure is not limited to this. In another embodiment, the terry structure is integrated with the main body 10 by an embroidery process. That is, the terry structure is embroidered on the inner side of the medical compression stockings. For the embroidered terry structure, only continuous tucks need to be embroidered here.

The material of the terry structure is natural fiber or man-made fiber, such as nylon and cotton yarn. The height, pressure and arrangement of the pile structure can be adjusted according to the shape, contour and pressure distribution of the lower limb.

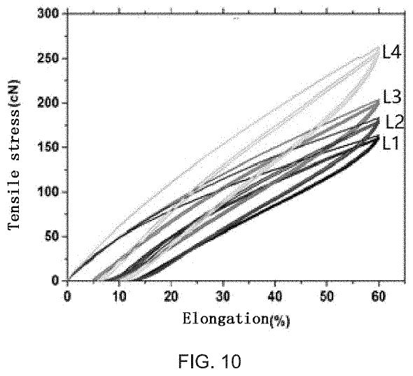

FIG. 10 is a schematic diagram of a relationship between tensile stress and elongation of medical compression stockings. The abscissa represents the elongation, the ordinate represents the tensile stress, L1 to L4 are the above-mentioned relationship curves of four kinds of medical compression stockings respectively, that is, L1 is a normal knitting structure, L2 is a tuck structure, and L3 represents a general knitting structure with terries, L4 represents a tuck structure with terries.

The Laplace formula, P=F/r, represents that the pressure is positively correlated with F (here, the tensile stress). According to FIG. 10, it can be seen that the tensile stress of the tuck form and/or the knitting structure with terries has been increased.

The form and structure of the auxiliary pressure part 20 are not limited to this. In another embodiment, the auxiliary pressure part 20 may include a gasket, and the gasket is sewn on the medial malleolus of a lower limb of the medical compression stockings.

The material of the gasket is silicone, the auxiliary pressure part 20 further includes auxiliary fabrics, the silicone is sandwiched between the main body and the auxiliary fabrics, area of the auxiliary fabric is greater than or equal to area of the silicone, and the auxiliary fabric is in direct contact with the medial malleolus of the wearer.

That is, in the embodiment, the auxiliary pressure part 20 is composed of a three-layer structure, the outer side is the fabric of the medical compression stockings, the middle is the gasket, and the inner side is the auxiliary fabrics, which is in contact with the skin.

Hereinafter, the structure of the pressure sensing part 30 of the medical compression stockings will be described in detail through a specific embodiment.

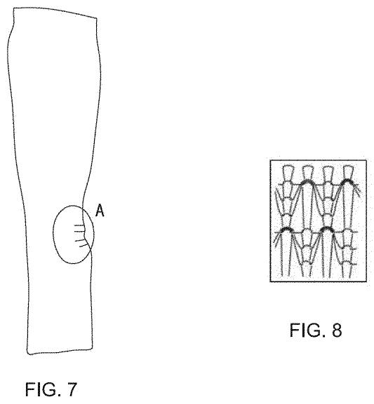

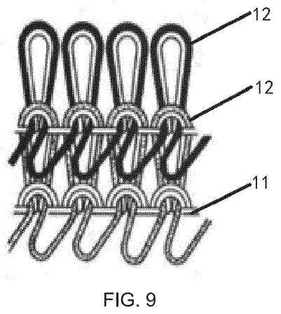

In the embodiment, as shown in FIG. 5 and FIG. 6, the conductive part may include a flexible conductive part 31, the flexible conductive part 31 is sewn into the main body 10 to be integrated with the main body 10, and the flexible conductive part 31 senses pressure changes through changes in resistance or capacitance.

The flexible conductive part 31 is a flexible conductive wire or a conductive elastic wrap yarn, and the flexible conductive part 31 is integrated into the main body 10 by means of loop formation, yarn addition or laid-in.

The conductive part further includes a connecting wire 32, and the flexible conductive part 31 is electrically connected to the signal receiving part 33 through the connecting wire 32. Further, it can also be electrically connected to the analysis and calibration system.

In the embodiment, the flexible conductive part 31 is used as the main part of the conductive part to be integrated into the medical compression stockings. It is easy to bend and deform, and the material is thin and elastic, which not only meets the wearer's comfort requirements, but also can withstand low mechanical resistance, and can be easy to put on and take off, hang and durable. Therefore, the requirements for wearing and pressure detection are met at the same time, and it is easy to manufacture.

The number of the conductive parts may be one or more, which are arranged at one or more positions of the main body 10, so as to monitor the pressure at the corresponding positions in real time. As shown in FIG. 5, the conductive parts can be located at B, B1, and C. The number and arrangement of the conductive parts can be defined according to the RAL-GZ 387/1 and FTTS-FP-148 standards. The conductive parts can be along the length direction. The conductive parts can be aligned and spaced along the length direction, and can be located on the inner side, outer side, front side and/or back side of the lower limb of the medical compression stockings.

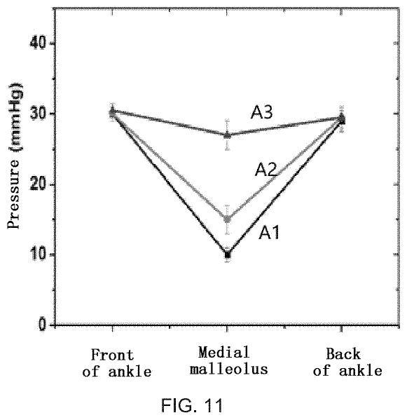

FIG. 11 is a schematic diagram of pressure distribution at multiple positions of medical compression stockings. The abscissa represents the different positions of the lower limbs, the ordinate represents the pressure, and A1 to A3 are above-mentioned relationship curves of three kinds of medical compression stockings. That is, A1 is a normal knitting structure, A2 is a tuck knitting structure, and A3 is a tuck knitting structure with terries. According to FIG. 11, at the position of the lower limb away from the medial malleolus, the pressure exerted by the above three knitting structures is about the same, while at the position of the medial malleolus, the pressure exerted by the knitting structure in the form of a tuck with terries is significantly greater than the other two structures, and the pressure of the medial malleolus is very close to the pressure of other positions of the lower limbs, that is, the pressure difference is small, and the pressure of the entire lower limbs is evenly distributed.

In addition, in the present disclosure, an auxiliary pressure part is integrated at an ankle of a lower limb of medical compression stockings. When worn, the auxiliary pressure part can properly fill hollow area between the medial malleolus and the tibia, and compensate the pressure of this part, so that the pressure from the compression stockings is transmitted to the ankle, so the pressure of the ankle is even, which can optimize the leg, especially the ankle, and avoid ulceration of patients with venous insufficiency. Moreover, the auxiliary pressure part is integrated with the main body, the two are inseparable, therefore, the auxiliary pressure part will not be displaced relative to the main body during wearing and putting on and off, and can always be kept at the position corresponding to the ankle of the lower limb, thus ensuring accuracy and convenience of use.

In addition, the medical compression stockings are also integrated with a pressure sensing part, so that the pressure applied by the medical compression stockings to the legs can be measured in real time, and the measured values have extremely high accuracy and reliability. Therefore, it is helpful for the wearer to monitor the comfort of the medical compression stockings.

Although the present disclosure has been described with reference to a few typical embodiments, it should be understood that the terms used are illustrative and exemplary rather than restrictive. Since the present disclosure can be implemented in various forms without departing from the spirit or essence of the invention, it should be understood that the above-mentioned embodiments are not limited to any of the foregoing details, but should be interpreted broadly within the spirit and scope defined by the appended claims. Therefore, all changes and modifications falling within the scope of the claims or their equivalents shall be covered by the appended claims.

1. Medical compression stockings, comprising:
a main body, formed by knitting yarn and inlay yarn;
an auxiliary pressure part, integrated with the main body and located at an ankle of a lower limb of the medical compression stockings, wherein the auxiliary pressure part is configured to be in contact with an ankle of a wearer; and
a pressure sensing part, comprising a conductive part and a signal receiving part, wherein the conductive part is integrated with the main body to sense pressure of corresponding part of the medical compression stockings, and the signal receiving part is configured to receive a signal from the conductive part and determine a pressure value of the corresponding part according to the signal.

2. The medical compression stockings according to claim 1, wherein, the knitting yarn and/or the inlay yarn are single-layer elastic wrap yarn.

3. The medical compression stockings according to claim 1, wherein, the knitting yarn and/or the inlay yarn are double-layer elastic wrap yarn.

4. The medical compression stocking according to claim 1, further comprising one or more additional auxiliary pressure parts each configured to directly contact a medial malleolus of the wearer.

5. The medical compression stockings according to claim 4, wherein, the auxiliary pressure part comprises a terry structure, and the terry structure is integrated with the main body by embroidering or knitting process.

6. The medical compression stockings according to claim 5, wherein, knitting yarn corresponding to the auxiliary pressure part is formed into a plurality of continuous tuck structures using a tuck method.

7. The medical compression stockings according to claim 6, wherein, along a warp direction, two to three tuck structures of the plurality of continuous tuck structures are continuously arranged, and along a weft direction, three to five tuck structures of the plurality of continuous tuck structures are continuously arranged.

8. The medical compression stockings according to claim 5, wherein, material of the terry structure is natural fiber or man-made fiber.

9. The medical compression stockings according to claim 4, wherein, the auxiliary pressure part comprises a gasket, and the gasket is sewn on a lower limb of the medical compression stockings corresponding to the medial malleolus of the wearer.

10. The medical compression stockings according to claim 9, wherein, the gasket comprises silicone, the auxiliary pressure part further comprises auxiliary fabrics, wherein the gasket is sandwiched between the main body and the auxiliary fabrics, wherein an area of the auxiliary fabric is greater than or equal to an area of the gasket, and the auxiliary fabrics is configured to be in direct contact with the medial malleolus of the wearer.

11. The medical compression stockings according to claim 1, wherein, the conductive part comprises a flexible conductive part, the flexible conductive part is sewn into the main body to be integrated with the main body, and wherein the flexible conductive part is configured to sense pressure changes through changes in resistance or capacitance.

12. The medical compression stockings according to claim 11, wherein the flexible conductive part is a flexible conductive wire or a conductive elastic wrap yarn, and the flexible conductive part is integrated into the main body by means of loop formation, yarn addition or laid-in.

13. The medical compression stockings according to claim 1, further comprising one or more additional conductive parts arranged at one or more positions of the main body.

14. The medical compression stockings according to claim 13, wherein the conductive part and the one or more additional conductive parts are arranged according to positions where pressure measurement is required.

15. The medical compression stockings according to claim 13, wherein, the conductive parts are located on inner side, outer side, front side and/or back side of the lower limb of the medical compression stockings.

16. The medical compression stockings according to claim 11, wherein, the conductive part further comprises a connecting wire, and the flexible conductive part is electrically connected to the signal receiving part through the connecting wire.

17. The medical compression stockings according to claim 1, wherein, the signal receiving part is also configured to compare the pressure value with a pressure threshold, and when the pressure value exceeds the pressure threshold, a reminder is issued to the wearer.
